(19) United States
(12) Patent Application Publication
(10) Pub. No.: US 2026/0355737 A1
(43) Pub. Date: Nov. 12, 2026

(54) FLOW ASSAY DEVICE FOR COLLECTING A BLOOD SAMPLE

(71) Applicant: DAKLAPACK EUROPE B.V., Lelystad (NL)

(72) Inventors: Dave Willem KLAASSEN, LELYSTAD (NL); Eryk Marcin TOMCZAK, LELYSTAD (NL)

(73) Assignee: DAKLAPACK EUROPE B.V., Lelystad (NL)

(21) Appl. No.: 19/130,847

(22) PCT Filed: Nov. 8, 2023
(86) PCT No.: PCT/EP2023/081136

§ 371 Date: May 16, 2025

(30) Foreign Application Priority Data
Nov. 18, 2022 (NL) 2033568

(51) Int. Cl. B01L 3/00; G01N 33/543

(52) U.S. Cl. CPC B01L 3/5023; G01N 33/54388; B01L 2200/025; B01L 2300/021; B01L 2300/069; B01L 2300/0825; B01L 2300/12

(57) ABSTRACT

A flow assay device for collecting a dried blood sample on a flow strip. The flow assay device has a tube for containing the flow strip. The flow strip is positioned inside the tube, in which a head portion of the flow strip extends outside the tube. A cassette is provided for housing the tube containing the flow strip. The cassette has a sample well for receiving a blood sample onto the head portion of the flow strip. The flow assay device is characterised in that at least 50% of a total length of the flow strip is contained in the tube which is beneficial in handling the collected blood sample. In another aspect, a pestle including a plug is provided for pushing the flow strip deeper into the tube.

The present invention relates to a flow assay device for collecting a biological sample, in particular a blood sample and a method, wherein use is made of such a flow assay device. In particular, the invention relates to a flow assay device including a flow strip inside a test tube in a cassette.

WO2020/251849 in the name of Siemens Healthcare Diagnostics Inc and published in 2020 discloses a sample device for separating and metering a plasma sample from a patient's liquid test sample. The sample device is usable in a point-of-care application. The sample device comprises a top portion with a sample channel for collecting a patient's liquid test sample. When the sample channel is formed from the securement of the top portion and bottom portion to one another, the sample channel is capable of drawing in a patient's liquid test sample when the sample device is placed in the collection position. The sample device contains at least one red blood cell capture membrane and at least one plasma membrane being placed in overlapping arrangement, such that the patient's liquid test sample may be drawn into the red blood cell capture membrane and the patient's plasma sample is drawn into the plasma membrane. Following a collection of a patient's liquid test sample, the sample device is then inverted into a sampling position for placement within a reaction vessel.

In an embodiment, the plasma membrane comprises an exposed portion which may have a surface area of about 20 mm² that holds a plasma sample of about 1 μL for use in an analyte detection and/or diagnostic assays. Once the exposed portion contains a pre-determined amount of patient's plasma sample, the exposed portion of the plasma membrane may be cut off before the sample device is placed in a reaction vessel in order to meet and ensure that the exposed portion contains and delivers a predetermined volume of a patient's plasma sample.

A drawback of this known method for collecting a liquid sample is that the placement of the sampling device into the reaction vessel may be vulnerable to failures due to inappropriate handling and possible contaminations. A more reliable processing of a sample containing membrane is desired.

EP3705042B1 in the name of Biodesix Inc. and published in 2020 discloses a sample separation device to enable field collection of whole blood, and separation of plasma from the blood cells without the need of an intervening step such as centrifugation of the blood specimen.

The sample separation device comprises a housing with a door, a blood sample separation medium, a mesh material and a desiccant.

WO2021/225446 in the name of Labonovum and published in 2021 discloses a grinder for use in a method of processing a piece of lateral flow paper. A sample container, e.g. a test tube, with a base part is provided for storing a blood sample provided on a piece of lateral flow paper. The base part has protrusions extending into a storage volume of the sample container. The grinder has a grinder storage volume with through holes providing a fluid connection through the grinder body and grinding protrusions at an outer surface of the grinder body.

In use, a piece of lateral flow paper comprising blood serum may be placed into and/or onto the bottom of the storage volume of the test tube. In order to fit the piece of lateral flow paper into storage, a pestle may be used to push the piece of paper entirely into the storage volume. The pestle may be automatically operated by a machine. Furthermore, a liquid such as a buffer liquid may be provided to the storage volume as well. Next, the grinder may be placed into storage volume.

A drawback of the disclosed collection device is that the processing of the lateral flow paper after a collection of a blood sample is vulnerable to failures. A more reliable processing of a collected sample is desired.

Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim of this application common general knowledge in the field relevant to the present invention.

The general object of the present invention is to at least partially eliminate the above mentioned drawback and/or to provide a usable alternative. More specific, it is an object of the invention to provide a flow assay device which contributes to a more reliable processing of a collected sample.

According to the invention, this object is achieved by a flow assay device according to claim 1.

According to the invention, a flow assay device is provided for collecting a biological sample, in particular a dried blood sample, on a flow strip. The flow assay device comprises a bibulous flow strip for absorbing and storing the sample. The flow assay device comprises a tube for containing the flow strip. The flow strip is positioned inside the tube, wherein a head portion of the flow strip extends outside the tube. Further, the flow assay device comprises a cassette for housing the tube containing the flow strip. The cassette has a sample well for receiving a biological sample onto the head portion of the flow strip. A user may drip some blood droplets at the sample well onto the head portion of the flow strip, where after the blood sample flows through the flow strip.

The flow assay device according to the invention is improved in that at least 50% of a total length of the flow strip is contained in the tube. In other words, at most 50% of the total length of the flow strip extends outside the tube. The tube contains the at least 50% of the flow strip. In particular, at least 70% of the total length of the flow strip is contained in the tube. Preferably, for blood separation, the flow strip extends at most 30 mm, in particular at most 20 mm outside the tube.

The containment of a main part of the flow strip in the tube warrants that the flow strip remains in the tube during handling by a laboratory operator or machine. The containment of the main part of the flow strip inside the tube is further beneficial in that after the collection of the biological sample, the flow strip can be pushed in a more reliable manner into the tube.

After collecting the biological sample at a point of care, the flow assay device can be transported to a laboratory for diagnosis of the biological sample. At the laboratory, in a method of handling a biological sample, the cassette can be opened by a laboratory operator or a laboratory machine to take out the tube which holds the flow strip. The tube with the flow strip can be removed completely out of the cassette or may remain connected to a cassette bottom, wherein only a cassette top of the cassette is removed.

In a next step, the flow strip is then pushed into the tube by using a pestle. The pestle may be a manual tool to push the flow strip into the tube by the laboratory operator, or may be a machine component for automatically pushing the flow strip into the tube, in particular to a tube bottom. Before pushing, only a relative small length of the flow strip extends outside the tube which eases the operator to push the flow strip completely into the tube. A possible free swinging of a flow strip end outside the tube is limited. The flow strip will easily be inserted completely into the tube and can easily be pushed to the tube bottom. Furthermore, due to the relative short extending portion, a possible risk of a contamination, or an unintentional touch of the flow strip end by an operator is reduced.

In many cases, in a method of handling a biological sample, in processing the flow strip at a laboratory, the head portion will first be shortened in a cutting step. Preferably, the tube holding the flow strip is completely removed from the cassette before cutting off the head portion of the flow strip. The relative short extending portion of the flow strip contributes in an ease for an laboratory operator to cut off this head portion. After shortening the head portion, an even shorter outside the tube extending portion remains. Herewith, the cutting step in a method of collecting a biological sample further improves the reliability of a next processing step in which the flow strip is completely inserted in the tube.

Preferably, the tube is a standard test tube, in particular a micro-tube. The standard test tube has normalised dimensions according to laboratory standards. Herewith, the test tube is usable in commonly provided laboratory equipment. Preferably, the tube is a classic test-tube consisting of a cylindrical tube wall and a closed tube bottom delimiting a tube inner space. Preferably, the tube is tagged by an identification tag, for example by a barcode or a quick response (QR) code. In particular, the cassette of the flow assay device comprises a view window for visualising the flow strip inside the cassette, such that a user can control the collection of the biological sample by viewing that a predetermined sample volume is collected. The flow strip may contain a strip mark for indicating that a sufficient sample volume is obtained. When the strip mark is passed by the sample flow during a collection, the user has a clear indication that the predetermined sample volume is obtained. Preferably, the view window of the cassette is configured to visualise the identification tag of the tube inside. The identification tag, e.g. a barcode, may be scanned by the user, typically a caregiver at a point of care, through the view window for registry of the collected sample.

The flow strip is a collection strip, in particular a lateral collection strip, e.g. a lateral flow paper, which collection strip is configured to absorb the biological sample. The collection strip may comprise a sheet of glass fiber. The collection strip for blood separation may be a LF1 strip of GE Healthcare for collecting just a few or even a single drop of whole blood. The collection strip for blood separation may be a MF strip for collecting a whole blood volume around 100 μL. The collection strip may be a cellulose paper, e.g. a Whatman cellulose paper. The biological sample is added to the flow strip from the sample well and laterally flows from the head portion along the collection strip. Preferably, the collection strip is configured to absorb a predetermined volume of biological sample, e.g. 200-500 μL, which may be required to carry out a particular analyte detection or diagnostic assay. Typically, the collection strip has a width of at least 10 mm, in particular the collection strip has a width between 10 and 25 mm, e.g. 12 mm, 17 mm or 22 mm.

Preferably, the flow strip is configured for a separation of plasma and blood cells from a blood sample. The flow strip may be formed by a plasma membrane and a blood cell membrane which may be adjacently positioned in a partly overlapping arrangement. Preferably, the blood cell membrane and plasma membrane are integrally formed as a one-piece item. Preferably, the head portion of the flow strip contains a filter zone for separating the blood sample. The filter zone is arranged to separate a blood plasma from blood cells of the blood sample. The blood cells may be sieved from the blood plasma flowing through the filter zone. Preferably, the filter zone is positioned outside the tube. After cutting of the head portion of the flow strip, substantially only blood plasma is received in the tube. In the cutting step, a blood cells containing portion of the flow strip may be removed, and a blood plasma containing portion of the flow strip may be collected in the tube.

The flow assay device is a point-of-care device which means that the collection of the biological sample can be carried out at home of a user, or a location of a care provider. The collected biological sample on the flow strip of the flow assay device, is called a dried biological sample, which means that the substantially the whole biological sample is absorbed by the flow strip without releasing sample liquid in the tube. The containment of the complete volume of the biological sample by the flow strip may prevent spoiling of biological sample e.g. caused by an adherence of sample liquid to an inner tube wall.

In an embodiment of the flow assay device according to the invention, the flow assay device further comprises a pestle for pushing the flow strip into the tube. Preferably, the pestle is a manual tool which is configured to be handled by a laboratory operator. The pestle may have a rod body provided with a finger grip at a proximal end and an enlarged portion, a push head, at a distal end. The push head may for instance be formed by a forwards pointing conical portion. In a method of handling a biological sample in the flow strip, the push head may be positioned at a tube opening and moved to the tube bottom while pushing the flow strip.

In an embodiment of the flow assay device according to the invention, the pestle comprises a plug. A pushrod is to be used in cooperation with the plug to move the plug to the tube bottom. The plug and the pushrod are separate items allowing the plug to remain in the tube after pushing the flow strip to the tube bottom. The plug keeps the flow strip at the tube bottom. The plug is beneficial in storing the biological sample inside the tube in which the whole flow strip is covered and protected by the tube.

The pushrod may be any conventional laboratory instrument which is suitable to push the plug through the tube. The pushrod may be a disposable and manufactured out of a plastic material, e.g. PP by injection moulding. The pestle may have a cylindrical shape corresponding with an inner shape of the tube, wherein a head portion of the pestle is closed to push the flow strip to a tube bottom.

Preferably, the pushrod is adapted to cooperate with the plug. The pestle comprises an assembly of a plug and a pushrod which pushrod is adapted to cooperate with the plug. In an embodiment, the pushrod as a separate item has a finger grip at a proximal end and a push portion at a distal end for pushing the plug. In a processing step of collecting the biological sample, the laboratory operator inserts the plug into the tube, whereafter the pushrod is used to push the plug towards the tube bottom.

Preferably, the plug is made of a silicon material. Preferably, the plug has a shuttle shape. The shuttle shape is beneficial in positioning and displacing the plug through an inner space of the tube. The shuttle shape of the plug includes a semi-spherical head portion and a tail portion.

An outer diameter of the head portion is adapted to an inner diameter of the tube to engage with the flow paper. Preferably, the head portion includes a least one through hole. The at least one through hole allows a later added buffer liquid to pass the plug to get in contact with the flow strip, e.g. to solve the biological sample contained by the flow strip. Preferably, the through-hole is positioned at a central region of the head portion. In addition or in an alternative embodiment, the head portion may further comprise at least one channel at an outer circumference to allow a passage of fluid.

An outer diameter of the tail portion is typically larger than the outer diameter of the head portion. The tail portion of the plug may comprise a plurality of freely extending tails positioned along a circumference of the head portion. The freely extending tails are preferably outwardly inclined to engage with an inner tube wall. The pushrod may have a push portion which is configured to fit in between the extending tails to rotatably engage with the plug. An operator may rotate the pushrod to rotate the plug to enhance the insertion of the flow strip in the tube.

In an embodiment of the flow assay device, the cassette has an oblong shape. The oblong shape is adapted to the elongated shape of the tube holding the flow strip. The cassette may be made by injection moulding. The cassette may be formed by a cassette top and a cassette bottom, wherein the tube containing the flow strip is held by the cassette bottom and covered by the cassette top. The cassette bottom and the cassette top are connected to each other.

The connection of the cassette bottom and cassette top is manually irreleasable by a user and configured to be loosened by a laboratory operator or a machine.

In an embodiment of the cassette of the flow assay device, the cassette bottom comprises a support member for supporting the head portion of the flow strip. The cassette top comprises the sample well for receiving the biological sample. The sample well is positioned opposite the head portion of the flow strip, in particular opposite the support member.

The sample well may be provided with a pierce member for piecing a user's finger which beneficially makes a separate blood lancet redundant. Preferably, the cassette top further comprises at least one view window for observing a collected sample volume. The view window may be formed by an elongated aperture in the cassette top. The view window is preferably formed by an open aperture. Alternatively, the view window is provided by a transparent material. Preferably, the tube provided with the identification tag is positioned behind the view window, such that the identification tag is readable through the view window. Beneficially, the identification tag of a standardised test tube can be used for identification of the sample assay device.

In an embodiment of the flow assay device, the cassette is made of a cardbox material. Herewith, the disposable cassette is easy recyclable. The cassette is box-shaped for housing the test and flow strip. Preferably, a sealable container, a bag or envelope, is provided in a sample collection kit according to the invention for storing and sending the flow assay device to a laboratory.

Further, the invention relates to a sample collection kit comprising a flow assay device according to the invention. The sample collection kit comprises sealable container, for example a sealable bag or envelope, for packaging the flow assay device to stand the flow assay device to a laboratory.

Further, the invention relates to a method for handling a biological sample, in particular a dried blood sample, captured on a flow strip of a flow assay device. In particular, in the method according to the invention, a blood plasma is separated from a blood sample. In particular, the method is a serological test method on a blood sample collected by filter paper. Typically, the method is carried out in a laboratory by a laboratory operator. The flow assay device according to the invention is used in the method. The method comprises a step of providing a flow assay device according to the invention having a flow strip inside a tube which subassembly of the tube with flow strip is contained by a cassette. In a step, a biological sample is received from a user which biological sample is collected by the user via a sample well of the cassette. The biological sample is absorbed in a flow strip inside the cassette. In a step, the cassette of the flow assay device is opened. A cassette top may be removed from a cassette bottom to expose the flow strip being partly positioned in the tube. The tube holding the flow strip may be taken out of the cassette. Optionally, in a step of the method, a head portion of the flow strip may at least partly be cut off. Preferably, a flow strip portion including a filter zone is cut off from the remaining portion of the flow strip to obtain a separated volume of the biological sample, in particular the blood plasma sample. In a step of the method, a pestle is inserted into the tube and the flow strip is pushed to a tube a bottom. Herewith, the flow strip is fully contained inside the tube and beneficially brought in a situation in which the flow strip is protected from the environment by the tube. By using the flow assay device according to the invention, due to the relative short extending head portion, it is convenient to push the flow strip by a pestle completely into the tube and preferably to the tube bottom.

In an embodiment of the method according to the invention, the pestle comprises a plug which is inserted into the tube, whereafter a pushrod is used to push the plug together with the flow strip into the tube and preferably to the tube bottom. Advantageously, the plug may serve as a stopper to keep the flow strip in the tube.

According to an aspect of the invention, the invention relates to a flow assay device for collecting a biological sample, in particular a dried blood sample, on a flow strip, wherein the flow assay device comprises a flow strip for absorbing and storing a biological sample, a tube for containing the flow strip, in which the flow strip is positioned inside the tube and wherein a head portion of the flow strip extends outside the tube, and a cassette for housing the tube containing the flow strip, wherein the cassette has a sample well for receiving a biological sample onto the head portion of the flow strip, wherein the sample well is aligned with the head portion of the flow strip, characterised in that the flow assay device further comprises a pestle for pushing the flow strip into the tube, wherein the pestle comprises a plug as a separate item which plug fits in a tube inner space.

The pestle comprising a plug is beneficial in that the plug may serve as a stopper and remain in the tube to keep the flow strip containing the biological sample inside the tube. Preferably, the plug is provided with a passageway, in particular a through hole in an axial direction or an outer flow channel allowing a laboratory operator to add a buffer liquid for solving the biological sample to the tube, while leaving the plug in position inside the tube.

Embodiments of the pestle are described above and illustrated in the detailed description hereafter.

The invention will be explained in more detail with reference to the appended drawings. The drawings show a practical embodiment according to the invention, which may not be interpreted as limiting the scope of the invention. Specific features may also be considered apart from the shown embodiment and may be taken into account in a broader context as a delimiting feature, not only for the shown embodiment but as a common feature for all embodiments falling within the scope of the appended claims, in which:

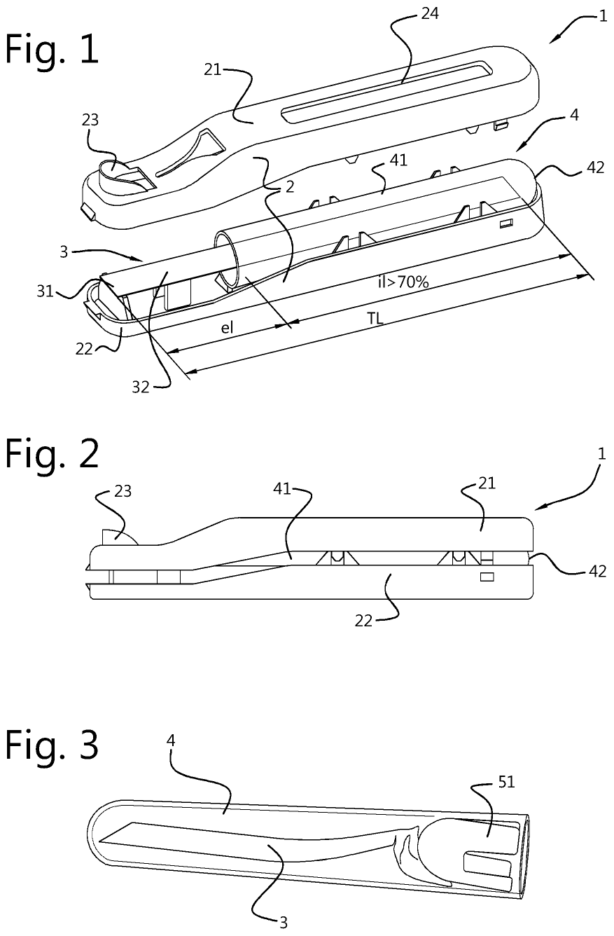

FIG. 1 shows an exploded view of an embodiment of the flow assay device according to the invention having a tube holding a flow strip inside a cassette;

FIG. 2 shows a side view of the flow assay device of FIG. 1;

FIG. 3 shows a flow strip which is put inside the tube by a plug of a pestle;

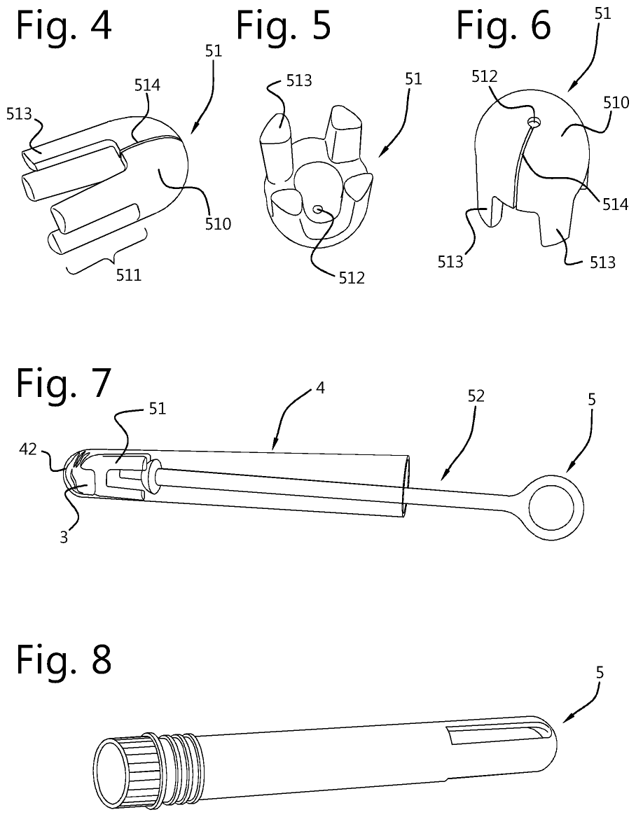

FIG. 4-7 show several perspective views of the plug of FIG. 3; and

FIG. 8 shows an alternative embodiment of a pestle having a cylindrical pestle body dimensions in correspondence with a tube inner space.

In FIGS. 1 and 2, a flow assay device according to the invention is denoted overall by reference numeral 1. Identical reference signs are used in the drawings to indicate identical or functionally similar components.

FIG. 1 shows an exploded view of an embodiment of the flow assay device 1 according to the invention. The flow assay device 1 is arranged to collect a biological sample, in particular a dried blood sample, on a flow strip, also called a collection strip. The flow assay device 1 has a cassette 2 for housing a subassembly of a flow strip 3 which is positioned inside a tube 4.

The cassette 2 has a cassette top 21 which is connected to a cassette bottom 22. The cassette 2 has a sample well 23 for supplying a biological sample, in particular a blood sample, to the flow strip 3. Further, the cassette 2 has a view window 24 allowing a user to view a progression of sample collection. When the user supplies sample droplets onto a head portion 31 of the flow strip 3, the sample liquid flows through the flow strip 3 which is visible through the view window 24. The flow strip 3 may have a strip mark at a predetermined position at the flow strip 3 and visible through the view window 24 to indicate to the user when a sufficient sample volume is collected.

The flow strip 3 is arranged to absorb a supplied biological sample. The flow strip 3 has an absorbing capacity which is configured to absorb a predetermined sample volume. For example, the absorbing capacity of the flow strip 3 may be at least 100 μL and at most 500 μL, preferably about 200 μL.

In particular, the flow strip is a blood collection strip. The blood collection strip is configured to collect a blood sample. In particular, the blood collection strip is configured to separate blood plasma from a supplied blood sample. The blood collection strip may include a filter zone 32 for receiving blood cells from the blood sample and to allow blood plasma to pass.

As shown, the flow strip 3 is contained in the tube 4. The tube 4 is a standard test tube, generally known as a micro tube. Typically, the micro tube has a volume of 1 to 5 mL, in particular the tube 4 has a volume of 5 mL. Here, the tube 4 may have a length of about 60 mm and a diameter of about 18 mm which is suitable to receive a flow strip having a width of 12 or 17 mm.

The dimensioning of the tube 4 is adapted to the length of the flow strip 3. The tube 4 houses at least 50% of the total length TL of the flow strip 3. A head portion 31 of the flow strip 3 is exposed outside the tube 4. In other words, the flow strip 3 has an inserted length iL which is positioned inside the tube 4, and an extending length eL which extends outside the tube 4. The extending length eL is at most 50% of the total length TL of the flow strip 3. Here, in the drawing, the internal length is at least 70%. Advantageously, the relative large enclosure of the flow strip 3 by the tube 4 contributes in a further processing of the collected sample.

Here, the head portion 31 contains the filter zone 32 for receiving the blood sample. The filter zone 32 is positioned outside the tube 4. In processing the biological sample, the head portion including the filter zone may be cut off from the flow strip 3 to process only a separated portion of the biological sample, in particular a blood plasma sample.

In further processing the biologic sample, the flow strip 3 is completely inserted in the tube 4. A pestle 5 is used by a laboratory operator or a rotary machine to push the flow strip 3 into the tube 4. Preferably, the pestle 5 is used to push the flow strip 3 deep into the tube 4, such that the flow strip 3 is pushed to the tube bottom 42.

As shown in FIG. 3, the pestle 5 may comprise a plug 51 for pushing the flow strip 3 into the tube 4. The plug 51 may be manually inserted into the tube 4 to push the extended portion of the flow strip 3 into the tube 4. Herewith, the flow strip 3 is completely contained inside the tube 4 and the plug 51 may serve as a stopper. Herewith, the flow strip 3 is contained for storage or further processing.

FIG. 4-6 show the plug 51 in further detail in several perspective views. The plug 51 may have a variety of shapes, for example ball or sleeve shaped, but is preferably shuttle shaped. The shuttle shaped plug 51 has a head portion 510 and a tail portion 511. The head portion 510 is semi-spherical. The tail portion 511 is formed by a plurality of tails 513.

As shown, the plug 51 may have a through hole 512. The through hole 512 is preferably centrally positioned. The through hole 512 is beneficial to allow a passage of a fluid, a liquid or air, along the plug. Air may be discharged when pushing the plug 51, or a liquid, in particular a buffer liquid, may pass the plug when supplied in preparation of a diagnostic step. Here, the plug 51 further comprises at least one channel 514 at an outer surface. The channel 514 extends in axial direction along the outer surface. Like the at least one through hole 512, the at least one channel 514 may allow a passage of fluid. Preferably four channels are equally spaced around the circumference.

As shown in FIG. 7, the pestle 5 may comprise a pushrod 52 to push the plug 51 together with the flow strip 3 deeper into the tube 4. Here, the flow strip 3 is pressed against the tube bottom 42. The pushrod 52 has a finger grip at a proximal end and an enlarged portion at a distal end. The enlarged portion forms a push head for pushing the plug 51 into the tube 4. Here, in use, the push head abuts against the tails 513 of the plug.

FIG. 8 shows an alternative embodiment of a pestle 5 for pushing a flow strip 3 into the tube 4. The pestle 5 has a cylindrical pestle body. At a proximal end, the pestle 5 is provided with a screw thread and a finger grip which allows a screw mounting of the pestle onto the tube. Herewith, the pestle can be used to push the flow strip 3 deep into the tube and in addition to close the tube.

Numerous variants are possible in addition to the embodiment shown in the figures. For example, in a variant of the illustrated embodiment of the plug, an alternative embodiment of the plug may lack the illustrated tails. The plug may for example be ball shaped.

Although the present invention has been described in detail, it will be apparent to those skilled in the art that various changes and modifications can be made without departing from the scope of the invention as hereinafter claimed. It is intended that all such changes and modifications be encompassed within the scope of the present disclosure and claims.

Further, it is remarked that any feature of the system according to the invention which is described in the embodiments and/or mentioned in the dependent claims is in itself considered patentable without any dependency to another presented feature. In particular, any measure presented in a dependent claim is also considered patentable without dependency of the independent claim. In particular, the feature of the pestle comprising a plug is mentioned as an aspect of the invention and considered independent of the feature in that at least 50% of a total length of the flow strip is contained in the tube.

Thus, in an aspect of the invention, a flow assay device is provided which comprises a flow strip for collecting a biological sample, which flow strip is held for at least 50% of its total length in a tube which is housed in a cassette. In another aspect of the invention, a flow assay device is provided which comprises a pestle including a plug for pushing a flow strip deeper into a tube, such that the whole flow strip is contained by the tube.

Reference signs list:

TL total length
iL inserted length
eL extending length
1 flow assay device
2 cassette
21 cassette stop
22 cassette bottom
23 sample well
24 view window
3 flow strip
31 head portion
32 filter zone
4 tube
41 tube wall
42 tube bottom
5 pestle
51 plug
510 head portion
511 tail portion
512 through hole
513 tail
514 channel
52 pushrod

1. A flow assay device for collecting a biological sample including a dried blood sample, on a flow strip, wherein the flow assay device comprises:
a flow strip for absorbing and storing a biological sample;
a tube for containing the flow strip, wherein the flow strip is positioned inside the tube and wherein a head portion of the flow strip extends outside the tube;
a cassette for housing the tube containing the flow strip, wherein the cassette has a sample well for receiving a biological sample onto the head portion of the flow strip, wherein the sample well aligns with the head portion of the flow strip,
wherein at least 50% of a total length of the flow strip is contained in the tube.

2. The flow assay device according to claim 1, wherein the flow strip is configured for a separation of plasma and blood cells from a blood sample, and
wherein the flow strip is formed by a plasma membrane and a blood cell membrane.

3. The flow assay device according to claim 1, wherein the head portion of the flow strip contains a filter zone for separating the biological sample including for separating a blood plasma from blood cells.

4. The flow assay device according to claim 3, wherein the filter zone is positioned outside the tube.

5. The flow assay device according to claim 1, wherein the flow assay device further comprises a pestle for pushing the flow strip into the tube.

6. The flow assay device according to claim 5, wherein the pestle comprises a plug which fits in a tube inner space, and
wherein the plug is a separate item which may remain in the tube after pushing the flow strip into the tube.

7. The flow assay device according to claim 6, wherein the plug has a shuttle shape including a substantially semi-spherical head portion and a tail portion.

8. The flow assay device according to claim 7, wherein the head portion includes a through hole positioned at a central region of the head portion.

9. The flow assay device according to claim 7, wherein the tail portion comprises a plurality of freely extending tails.

10. The flow assay device according to claim 6, wherein the plug is made of a silicon material.

11. The flow assay device according to claim 1, wherein the cassette has a view window for determining a collection of a sufficient volume of biological sample along a length of the flow strip.

12. The flow assay device according to claim 11, wherein the view window enables a detection of an identification tag of the tube, in particular a barcode or QR code.

13. The flow assay device according to claim 1, wherein the cassette has an elongated shape including a cassette bottom and a cassette top which are connected to each other.

14. The flow assay device according to claim 1, wherein the cassette is made of a cardbox material.

15. The flow assay device according to claim 1, wherein the tube is a standarized test tube for general use in a laboratory, and
wherein the tube is a micro-tube.

16. A sample collection kit comprising the flow assay device according to claim 1, wherein the sample collecting kit further comprises a kit package including a sealable container for sending the flow assay device by a postal service to a laboratory.

17. A method for handling a biological sample, in including a dried blood sample, on a flow strip of a flow assay device, wherein the method comprises:
providing the flow assay device according to claim 1 comprising a flow strip inside a tube positioned inside by a cassette;
receiving a biological sample from a user which biological sample is collected via a sample well of the cassette and absorbed in the flow strip inside the cassette of the flow assay device;
opening the cassette of the flow assay device;
inserting a pestle into the tube;
pushing the flow strip into the tube to the tube bottom.

18. The method according to claim 17, further comprising:
removing a pushrod of the pestle out of the tube, while leaving a plug of the pestle inside the tube.

19. The method according to claim 18, further comprising:
adding a liquid to the tube while leaving the plug in the tube.

20. The flow assay device according to claim 2, wherein the head portion of the flow strip contains a filter zone for separating the biological sample, for separating a blood plasma from blood cells.
